(19) United States
(12) Patent Application Publication
(10) Pub. No.: US 2022/0230737 A1
(43) Pub. Date: Jul. 21, 2022

(54) Personalizing the Experience for the Senior Living Resident Through Automated Data Collection, Workflow Tools, and Actionable Insights

(71) Applicant: TSOLife, Inc., Tampa, FL (US)

(72) Inventors: David Sawyer, Tampa, FL (US); Stella Parris, St. Petersburg, FL (US)

(73) Assignee: TSOLife, Inc., Tampa, FL (US)

(21) Appl. No.: 17/579,804

(22) Filed: Jan. 20, 2022

Related U.S. Application Data
(60) Provisional application No. 63/139,716, filed on Jan. 20, 2021.

(51) Int. Cl. G16H 40/20; G16H 10/65

(52) U.S. Cl. CPC G16H 40/20; G16H 10/65

(57) ABSTRACT

The invention relates to a system that in one form is a data collection system for use in various senior care facilities that utilizes data collection through automated means such as digitized records, voice recognition, video capture, smart forms and a cloud-based technology platform. The instant invention uses AI to translate audio information to solve the problem faced by senior living communities to collect reliable data.

### CROSS-REFERENCE TO RELATED APPLICATIONS

This application claims the benefit of U.S. Provisional Patent Application No. 63/139,716 filed on Jan. 20, 2021, which is incorporated by reference herein in its entirety.

### COPYRIGHT STATEMENT

A portion of the disclosure of this patent document contains material that is subject to copyright protection. The copyright owner has no objection to the facsimile reproduction by anyone of the patent document or the patent disclosure as it appears in the Patent and Trademark Office patent file or records, but otherwise reserves all copyright rights whatsoever.

Trademarks used in the disclosure of the invention, and the applicants, make no claim to any trademarks referenced.

### BACKGROUND OF THE INVENTION

### 1) Field of the Invention

The invention relates to the field of systems used to capture complete and accurate data resident profiles.

Currently, traditional resident profiles are collected by the facility staff using interview and hand written documentation collected from either staff, family or the resident. These methods result in missing information being incorporated into the resident profile due to the facility staff not recording or recording inaccurate information. The result is that the resident profile does not contain important information which can be used to provide increased awareness with respect to resident interest and background which results in missed opportunities for social interaction and overall fulfillment. The lack of complete and accurate data in senior living communities is a major issue affecting the facility management, residents and the caregiving staff.

Electronic Health Records (EHR) may provide task reminders, but they are not automated. These systems rely on staff to enter the relevant information or task and assigning the task to be completed. This requires a number of manual tasks to achieve the inputting of the data. The manual inputting of information leads to errors and requires a substantial amount of time to record as well as being difficult to retrieve.

Accordingly, there remains a need in the art for a simple and accurate method to collect accurate patient data and provide a simple means to retrieve the patient data.

### BRIEF SUMMARY OF THE INVENTION

The instant invention in one form is directed to a data collection system for use in senior care facilities which utilizes data collection through automated means such as digitized records, voice recognition, video capture, photographs, smart forms and a cloud-based technology platform. The instant invention also uses Artificial Intelligence (AI) to translate audio information and loaded it into the appropriate forms and data base. The AI process solves the problem faced by senior living communities which is to collect reliable resident specific data with minimal labor input. The instant invention onboarding process for new residents creates a digital record such as a database or forms which frees the caregivers and administrative staff members of the facility from the traditional ineffective paper based systems currently used.

The instant invention is capable of storing and analyzing the data using a matching algorithm which is capable of analyzing the resident profiles and finding other resident profiles which have matching histories, interests and backgrounds.

The instant invention recommendation engine essentially makes recommendations to the staff on how to provide a better experience for the residents. The algorithms take into consideration the resident's profile data, quality of life scores over time, actionable insights feedback, and event attendance. The concept of actionable insights as used in the specification is targeted information with regards to a specific resident so the management and staff have timely and relevant notifications about how to engage residents individually. They are structured by the individual resident and provide information about a resident combined with an action to take based on that information.

The instant invention in another form provides a means to match resident and caregivers such that a caregiver's interests are aligned to the resident's interests.

The instant invention in another form provides senior living facility management with a means to improve the experience of the residents by helping the care provider to capture complete and accurate data resident profiles.

The instant invention in another form provides a secure mechanism for collecting the resident information. The personal data collect by the system is transferred to the system for processing utilizing secure data transmission methods.

In a first implementation of the invention, is a data collection system for use in senior care facilities which utilizes data collection through automated means such as digitized records, voice recognition, video capture, photographs, smart forms and a cloud-based technology platform to create an accurate resident profile.

In a second aspect, the AI process that is integrated into the data collection system allows the user such as the caregiver or administrative staff the ability to collect information and it automatedly populates the database of smart forms used to store the information. The AI process also enables the user to collect the information using a variety of mechanisms such as but not limited the scanning hand written or typed information, audio and visual information and loaded it into the appropriate forms and database. The system utilizes translation of written information using Optical Character Recognition (OCR), audio files by voice to text software and smart forms to link photographs and videos to the appropriate smart form and database.

In another aspect, the instant invention can provide actionable insight to the caregivers and staff so they can have meaningful interaction with the resident being cared for.

These and other objects, features, and advantages of the present invention will become more readily apparent from the attached drawings and the detailed description of the preferred embodiments, which follow.

### BRIEF DESCRIPTION OF THE DRAWINGS

A further understanding of the nature and advantages of particular embodiments may be realized by reference to the remaining portions of the specification and the drawings, in which like reference numerals are used to refer to similar components. When reference is made to a reference numeral without specification to an existing sub-label, it is intended to refer to all such multiple similar components.

FIG. 1 shows a high level flow chart of the instant invention;

FIG. 2 shows a quality of life recommendations flow chart;

FIG. 3 shows a digitized resident profile screen of the instant invention;

FIG. 4 shows a typical quality of life data screen;

FIG. 5 shows a typical activity calendar data screen;

FIG. 6 shows a typical screen for feedback survey mechanism for the actionable insights;

FIG. 7 shows a typical screen for backend audio recordings and photos that are submitted in the mobile application that appear in the instant invention workflow;

FIG. 8 shows the typical screen for backend of the instant invention showing the edit transcript screen where the audio files can be cut into individual stories;

FIG. 9 shows the typical screen for backend of the instant invention showing that the stories are individually reviewed and edited to ensure quality;

FIG. 10 shows the typical screen for backend of the instant invention showing that the stories are matched with photos;

FIG. 11 shows the videos submitted through the mobile application also appear in the instant invention workflow;

FIG. 12 shows the schematic of the instant invention;

FIG. 13 Quality of Life Summary for an individual resident dashboard;

FIG. 14 Community Quality of Life summary dashboard; and

FIG. 15 Corporate Quality of Life summary dashboard.

Corresponding reference characters indicate corresponding parts throughout the several views. The exemplifications set out herein illustrate embodiments of the invention and such exemplifications are not to be construed as limiting the scope of the invention in any manner.

### DETAILED DESCRIPTION

While various aspects and features of certain embodiments have been summarized above, the following detailed description illustrates a few exemplary embodiments in further detail to enable one skilled in the art to practice such embodiments. The described examples are provided for illustrative purposes and are not intended to limit the scope of the invention.

In the following description, for the purposes of explanation, numerous specific details are set forth in order to provide a thorough understanding of the described embodiments. It will be apparent to one skilled in the art however that other embodiments of the present invention may be practiced without some of these specific details. Several embodiments are described herein, and while various features are ascribed to different embodiments, it should be appreciated that the features described with respect to one embodiment may be incorporated with other embodiments as well. By the same token however, no single feature or features of any described embodiment should be considered essential to every embodiment of the invention, as other embodiments of the invention may omit such features.

In this application the use of the singular includes the plural unless specifically stated otherwise and use of the terms “and” and “or” is equivalent to “and/or,” also referred to as “non-exclusive or” unless otherwise indicated. Moreover, the use of the term “including,” as well as other forms, such as “includes” and “included,” should be considered non-exclusive. Also, terms such as “element” or “component” encompass both elements and components including one unit and elements and components that include more than one unit, unless specifically stated otherwise.

Lastly, the terms “or” and “and/or” as used herein are to be interpreted as inclusive or meaning any one or any combination. Therefore, “A, B or C” or “A, B and/or C” mean “any of the following: A; B; C; A and B; A and C; B and C; A, B and C.” An exception to this definition will occur only when a combination of elements, functions, steps or acts are in some way inherently mutually exclusive.

As this invention is susceptible to embodiments of many different forms, it is intended that the present disclosure be considered as an example of the principles of the invention and not intended to limit the invention to the specific embodiments shown and described.

The use of the term community, facility, senior living community, care facility, nursing facility, nursing home, independent living facility and assisted living facility are used interchangeably and are meant to mean a facility where individuals live under a common management structure.

The use of term media file, audio file and video file are used interchangeably to refer to a media file that has an audio component.

The use of the term application, module or app as used within the specification is meant to mean a computer application and such computer application can run on any smart device or computer.

The use of the term Application Programming Interface (API) as used within the specification is meant to mean a digital connection between computers or between computer programs.

The use of the term resident, patient and individual is used interchangeably to refer to an individual living in a facility.

The use of the term staff, management, caregiver is used interchangeably and is meant to mean any individual that works in the facility.

The use of the term resident profile is meant to mean any resident specific information store either electronically or in paper form.

The use of the term system, instant invention, platform and process is meant to mean any a software system for residents in a senior living facility for determining actionable insights and recommending personalized care.

The term IP address as used in the specification is meant to mean a numerical label such as 192.0.2.1 that is connected to a computer network that uses the Internet Protocol for communication. An IP address serves two main functions: network interface identification and location addressing.

The term virtual private network is an arrangement whereby a secure, apparently private network is achieved using encryption over a public network, typically the internet.

The term session as used in this specification is meant to mean communication between two computer or smart devices using the internet protocol.

Prior to a discussion of the preferred embodiment of the invention, it should be understood that while the features and advantages of the invention are illustrated in terms of an automated system that utilizes digital computing systems to analyze and record data that a number of other systems both comprised of dedicated processing devices or general purpose processing devices can be utilized with the instant invention.

Currently in senior living communities, resident data, if collected at all, it is captured in a paper based process. There is generally a lack of standardization in how data is collected within corporations and across the industry as a whole. Staff at senior living communities have paper forms that they request the resident or their family fill out. The response found in the forms therefore, can be provided either by) a family member, the resident themselves, or a staff member on behalf of the resident.

In many cases, the form or forms are sent home with a family member as part of the paperwork to be completed at, or prior to moving into the facility. Generally, the forms are returned with incomplete information if they are returned at all.

The forms once returned to the community are filed using traditional paper based office practices which make them difficult to access and impossible to access in real time. If a staff member wants to know any information on a specific resident or a group of residents, they have to search the paper based records for the information and in many cases the specific information desired is missing. The current practices include:
a. writing with pen/pencil on paper;
b. typing information into a computer, tablet, or smartphone;
c. not collecting the data at all.

This information is important to the caregiver and resident relationship. If the information is accurately collected it provides helpful information about the resident to the caregiver/staff which makes the resident life at the facility more enjoyable and productive. When the information is unavailable or nonexistent, then the caregivers and staff that are working directly with residents must rely on their own knowledge of the resident that they have accumulated from conversations with the resident. While in many instances this informal system works, it does so until the system breaks down and the knowledge is lost. Whenever there is staff turnover, or at the end of a particular shift due to staff changes, information that is not written down is subject to being lost or forgotten. Since staff turnover is high in senior living communities, the knowledge that has been collected by one individual and not properly recorded is frequently lost and new staff or caregivers have to start over and as a result do not have the historical information to assist the resident. This disconnect in current processes results in data that is not being collected, and when it is, it is not ending up in the hands of those who need to know this information.

When data is collected in the paper form, the staff is asking the resident the question. For example, a staff member may ask the resident to “Tell them about their faith,”. The staff member then writes down the resident's response to the question while the resident is giving their response. People, in general, can talk faster than one can write, so even if the staff member is writing as quickly as possible, some information or details will still be missed. An example is that the resident tells the staff member a great story about how they came to their faith, but all that ends up on the form is a truncated note such as the resident is “Catholic.”

The paper based systems result in the care facility having very little information to operate on. The senior living operators continuously struggle to deliver on the resident experience that they want to provide. Repeatedly the residents of these communities experience social isolation, loneliness, depression, poor wellbeing, and low Quality of Life (QoL) which is associated with a range of negative physical health problems and may even result in reduced longevity.

Additionally, long-term care regulations require personalized care to be provided to residents. The delivery of personalized care becomes increasingly difficult with caregiver turnover which can be as high as 36.63%. When a caregiver leaves, knowledge is lost and providers struggle to maintain the personal caregiver-resident bonds that contribute to consistent care and positive customer satisfaction.

The instant invention essentially allows senior living communities to provide personalized care on a large scale and provide the caregivers important information with regards to the residents under their care. The instant invention provides communities the tools to enable their staff to track, monitor, and improve personal care and attention.

The instant invention resolves the issues of lost or missing data in the resident profile and provides the resident and facility the following unique features and benefits:
a. Data collection module—data is collected using media file audio recordings which eliminates the need for note taking of filling out forms. The instant invention uses Artificial Intelligence (AI) to transcribe the information and populate the smart forms. The media file audio files collected are automatically sent through a speech-to-text process to transcribe the media file audio to text after being submitted. The instant invention can also segregate the resulting text based on who is speaking whether it is the interviewer or interviewee. Data is only entered once, and it is done automatically and then accessible to everyone within the organization through the use of the cloud, client and mobile applications.
b. Speech-to-text Application Programming Interface (API) to transcribe the media file audio to text. The instant invention utilizes a speaker diarization feature such as Google's Diarization System, which divides the transcript based on who is speaking, whether the interviewer or interviewee (Speaker diarization is the process of partitioning an input audio stream into homogeneous segments according to the speaker's identity).
c. Smart forms module—The instant invention uses the audio software which locates the correct data in the media file audio files and then the instant invention automatically fills out the forms by placing the correct data in the correct form field.
d. Digital resident profiles module—provides readily available information from the cloud server using either the instant invention mobile application or client application on a remote computing device application.
e. Resident matching module—the instant invention matches residents based on common interests and experiences.
f. Recommendation engine module if the instant invention powers a Calendar module, Quality of life Recommendation module, and Actionable Insights module—the instant invention provides information extracted from the data that is populated in the resident profile to provide updates and recommendations for the resident using the instant invention calendar, Quality of Life recommendation list and actionable item list.
g. Inclusion score module—the instant invention formulates an inclusion score which looks at the events on a calendar for a user defined specified timeframe and calculates the percentage of residents that have an interest in the specific event on the calendar.
h. Timely delivery of data and feedback module—the instant invention provides a method to provide both the caregivers and the resident with timely information which they can use to improve the resident experience at the facility.
i. Dashboard module—the dashboards and analytics at both the community level, and at a corporate level for corporate level management are structured so that they can see their data in aggregate across all their communities.
j. The instant invention also provides the resident with a resident module which provides a resident with their own dashboard and information so they can review and track their events and calendar. This is available to them either from a client application on a connected computer system or mobile device.

The benefits of the instant invention over current processes are:
a. The automated data entry leads to time savings;
b. The information is collected using media file audio recording instead of manual transcription. The audio is also used to create life stories using the instant invention backend system. The audio is sectioned and matched to text, creating a story wall; and
c. The instant invention smart forms eliminate the need for staff to search through paper records to identify important information. The instant invention also eliminates the need for staff to manually enter the data into existing systems.

The instant invention provides an automated system for collecting information. The staff in the senior living communities conduct an interview with a resident and record a media file and associated audio file of the interview. The instant invention mobile application is designed to facilitate the recoding of resident information. A benefit of the instant invention is that the interview can be conducted in one sitting or multiple settings because the instant invention can combine files and analyze them and extract pertinent data and use the data to update the resident profile.

The mobile application is constructed so that it is easy to use. It is designed to provide a high contrast and simplistic view which makes it easy for staff to use. The instant invention can also utilize a specialized mobile device which is optimized for the interview process and has enhanced media file processing for both audio and video data. This specialized mobile device would be used by the interviewer when they interview a resident and it could have any of the following features:
a. Enhanced microphone
b. Enhanced video recording features
c. Dual head set and microphone so the interviewer and resident can have their own head set to make the interview easier to conduct.
d. Dedicated to a single application which provides increase security and eliminates the possibility of data being loss or not uploaded properly to the cloud application.
e. Dedicated IP address which is recorded in the cloud or dedicated server system which limits communication with the cloud or dedicated server system based on recognized IP address.

The Users of the mobile application when recording an interview simply initiates the record function using the mobile application to record, has the conversation with the resident, initiates the save function which saves the recording. The recording is then transferred by the instant invention and transcribed, so the information gathered during the interview is automatically populated in the forms or data structure where it needs to be. The information can be accessed in the resident profile, dashboard chart, in an actionable insights message, or as a recommendation on the activity calendar.

The instant invention provides a means for the senior care facility to collect increased information content and better quality data.
a. The increased information translates into the facility being able to deliver personalized care that can be delivered at a high standard and is scalable to large numbers of residents;
b. Senior living management has insights into patient needs and can manage accountability for programming at the corporate level; and
c. Facilities see measurable increase in data collected compared to the existing paper forms. Residents are more comfortable with the oral process which results in a conversational exchange yielding more information and better quality data. Prior to using the instant invention when an interview was conducted during the onboarding process, the amount of data collected was limited by how much information the staff member could write down while the resident is describing their history and providing relevant information.

The instant invention creates data that is actionable:
a. Once the data is collected, the instant invention uses unique and effective ways of disseminating the data so knowledge ends up with those who need it to do their job effectively.

The instant invention provides an enhanced resident experience:
a. A benefit of the process is a more personal, connected, and tailored-to-the-resident experience.

Resident profiles are found in a community's resident directory in the instant invention administration portal. The staff can access the resident information online using their digital resident profile and download and print the profiles if they wish. These profiles hold all the resident data and are available to the staff within the facility by accessing the cloud data using the mobile application.

Essentially, the resident profile is a digital version of the original paper form the staff currently uses and would have filled out by hand. The instant invention eliminates that paper form and creates a digital version of the form or forms which is populated from the information found in the media file audio of the interview(s). Therefore, the result is a digital, automatically filled out database that is presented in form format to the user, and contains all the information from the interview. This provides global knowledge to the senior care facility staff.

The instant invention facilitates matching residents on common interests and experiences. The information that is extracted from the media file audio recording of the interview can be used in the matching algorithm which is an intelligent subsystem of the instant invention. The matching algorithm has rules to help find residents with one-to-one field matching. The system prioritizes matches that will actually form a connection and bond. For example, two residents could both have marked “Yes” to the question, “Do you have military experience?” These residents do indeed have this fact in common, but even with that data match, on its own, it does not lead to an expansive conversation that would generate a fruitful bond. The algorithm then compares whether two residents were in the same branch of the military and even had some overlapping years of service. The algorithm can pull information from all the information collected on the residents and is able to mine the data in the resident's profile to develop a more detailed match that could lead to a more likely bond between the two residents. The instant invention then prioritizes the match and gives the match a weighted score.

The ability of the resident matching algorithm to compare data collected with regard to a resident with the data of other residents and staff members in the community provides a means to match both residents and staff to improve the personal interactions. The instant invention can compare and identify what the resident has in common as far as life experiences and interests. The instant invention can then rank a resident match based on the matching parameters which are user definable and can provide the community the top resident matches and top staff matches for each resident. Preferably the top resident match is limited to between 2 -15 matches, and the top staff matches for each resident is limited to between 2 -15 matches .This allows the facility to help increase the social interaction between residents and staff making for a more productive and enjoyable experience.

The matches can indeed be used to form bonds and create friendships. It can also be used to:
a. design tailored activities.
b. build small dining groups during the time of COVID-19 when group dining is prohibited.
c. choose who will be on the welcome committee for a new resident.
d. decide what residents a prospect should meet on a community tour.

Additionally, as noted earlier, the instant invention has a recommendation engine which is part of the system. Recapping the recommendation engine is essentially used to make recommendations to the staff on how to provide a better experience for the resident. The recommendation engine algorithms take into consideration the resident's profile data, Quality of Life scores over time, actionable insights feedback, and event attendance, if provided. The actionable insights tool empowers staff to provide a personalized resident experience, combating social isolation and loneliness and mitigating the knowledge loss of caregiver turnover, saving long-term care providers money and allowing staff to effectively deliver personalized care.

The instant invention Quality of Life process helps measure, track, and improve resident quality of life. This is accomplished by:
a. Measuring and tracking resident Quality of Life scores; focusing on a metric that matters to the resident and the facility.
b. Planning activities and engagement around what the residents need.
c. Demonstrate a ROI on investing in programming for residents.

It should be noted that senior living communities and software providers currently focus on Dimensions of Wellness' to ensure that activities offered at the community are varied and cover areas such as Purpose, Social, Physical, or Spiritual. The instant invention Quality of Life (QoL) subsystem is different in that it is based on a validated measure of Quality of Life, providing a validated measure for communities to plan activities to improve resident outcomes while still providing communities a marketable offering.

Once the resident QoL assessment data is inputted, it immediately informs the resident's QoL Summary on their resident details page. The instant invention creates a bar chart showing what the resident scored in each area (Leisure, Creativity, Learning, Friendships, How I View Myself, or How I View My life) and a pie chart displaying what the resident's total quality of life score is out of the maximum score of 100. However, any appropriate scale is applicable.

The instant invention technology is used to collect assessments in addition to the QoL assessment. A typical assessment display is shown in table 1. These assessment scores are extracted using the smart form technology and stored on the instant invention database systems. These assessments can be for a variety of health outcomes, satisfaction scores, net promoter scores, etc.

### Assessments

TABLE 1
Add Assessment

| Date | Assessment Type | Assessment Detail |
|---|---|---|
| Dec. 31, 2020 | Quality of Life | View Details |
| Dec. 31, 2020 | Wellness Snapshot | View Details |
| | Survey | |
| Dec. 31, 2020 | Net Promoter Survey | View Details |

Once the assessments have been collected, the instant invention's Artificial Intelligence (AI) modules and recommendations engines are then able to make recommendations to help the staff at the community to improve outcomes and scores for the residents and therefore improve resident quality of life, health outcomes, satisfaction scores and the quality of personalized care delivered to the resident.

If a resident scores low on a particular assessment or part of an assessment, the system is flagged to create recommendations to improve that specific assessment. The Artificial Intelligence (AI) modules looks at what data that has been collected with regards to a resident and what scores or assessments need improvement. The system then develops recommendations and phrases the recommendation in several ways, using research-backed techniques for social engineering and behavior change techniques. The instant invention system by using Artificial Intelligence (AI) modules over time gains enough information to know what the most effective wording is for each resident and the wording most likely to be delivered by the staff to achieve the desired outcomes.

These recommendations are then delivered as actionable insights to the staff at the community as a text message, email, notification on the instant invention mobile application, or popup in a computer software platform. The notification method is configurable and depends on the delivery method the community has selected for the actionable insights. The system seeks to improve assessment scores over time delivering multiple insights targeting the same problem area.

Looking at the instant invention calendar feature. The calendar feature can be used by communities to plan their calendar of events and activities for their community. Senior living in general is moving away from what it has done in the past, where communities offer bingo every day and spend a lot of time focusing on getting as many residents to an event as possible. Senior living operators are realizing that doing the same thing every day is not going to attract the residents and that a resident can be perfectly engaged and happy in a community but never show up to any events. When planning the activities calendar and community engagement, staff has to provide structure and variety, while keeping resident interests in mind. The instant invention calendar feature uses the community's resident data to make recommendations about what events to offer.

The instant invention also provides an inclusion score for the month, week, and day view of the calendar to demonstrate what percentage of the residents have their interests represented on the community calendar. Higher inclusion scores indicate the activity calendar is being created with resident interests in mind.

The instant invention actionable insights provide notifications enabling staff in senior living communities to leverage their resident data to provide a personalized resident experience. Actionable insights are delivered via email, notification, or retrieved from a newsfeed which can be accessed by the mobile application. To be an actionable insight, the recommendation must be insightful, leveraging a piece of resident data, and actionable, meaning it recommends an interaction with a resident that staff can easily complete.

The instant invention provides the actionable insights to the management and staff which are timely and relevant notifications about how to engage residents individually. They are structured by the individual and provide information about a resident combined with an action to take based on that information. Some examples include:
a. “Today is Veteran's Day! Remember to thank John Smith for his service in the Navy.”
b. “Today is Martha Smith's birthday. Wish her a Happy Birthday!”
c. “Mark Smith's favorite sports team, the Tampa Bay Rays, has a game today. See if he is interested in watching the game.”
d. “Pamela Smith prefers to take her medication with apple sauce. Try to accommodate when administering medication.”

Most knowledgeable caregivers believe that a resident in a senior living community should have several personalized interactions per day. The instant invention is a tool to help fuel these interactions.

The instant invention provides two types of insights: Community Newsfeed Insights and Tailored Insights.

Community Newsfeed—The purpose of these insights is to help staff in a community personalize the experience for residents, so the residents enjoy a good customer experience. These insights are date specific and can be delivered by any staff member within the community. These insights are broad, such as a birthday or anniversary reminder, and can be delivered multiple times by different staff.

Tailored Insights—The purpose of these insights is to help staff personalize their individual interaction with residents. These insights are tailored to both a specific resident and a specific staff member and are designed to be delivered at a specific day and time. To identify the correct timing, the instant invention integrates with a community's scheduling software. Additionally, tags such as QR codes, Bluetooth, Zigbee and Bluetooth low energy tags can be added outside each resident door to facilitate staff with more of a fluid schedule. These can be read by the specialized mobile device and application. Tailored insights are also used to ensure specific insights are only delivered once.

The instant invention provides a feedback mechanism which is for the actionable insights. Staff can mark the actionable insights complete, and then are prompted to complete a quick survey about how the actionable insight was received by the resident. If the actionable insight was poorly received, the system artificial intelligence module will learn not to recommend that insight again to that resident. For example, the system recommends the staff to wish a resident who is a veteran a Happy Veteran's Day, but the resident actually had a terrible time in the Navy, and never wants to talk about it, the staff member can log this feedback, and the system will no longer recommend that insight for that resident.

The instant invention provides the facility staff with customer dashboards that allow the staff to gain a deeper understanding of who is in their community, answer specific questions they have, and provide accountability.

For example, at the building level, the staff may schedule Mass and wants to make sure to invite all their Catholic residents. They can simply go to the religion chart in their dashboard, click the Catholic section of the chart, and immediately see a list of all of their Catholic residents, which they can view and print if they want. To gather this information, prior to the instant invention, a staff member would need to sit and manually flip through their binder of profiles which is time consuming and error prone.

The instant invention allows the facility and care giving staff to effectively utilize data and put the information into action as:
e. event recommendations when assembling the community calendar
f. actionable insights and messages to staff
g. recommendations to improve resident quality of life
h. dashboards displaying insights for the community

Additionally, the instant invention provides a more personal, connected, and tailored resident experience.

An example of this is a senior community has a new resident, Jane. Upon moving in Jane is interviewed using the instant invention mobile app by a staff member. She is asked questions about her life experiences, preferences, and interests.

That conversation is automatically uploaded and the instant invention artificial intelligence (AI) transcribes the interview and creates a digital resident profile for the resident's information.

The instant invention then populates the data into the smart forms and the system can identify who would be potential friends for Jane in the community by matching interests.

The instant invention can also notify the staff and invite Jane to community events that she has interests in.

When a caregiver goes to meet Jane, they get a push notification to their phone or specialized mobile device with a prompt about how to personalize the interaction with her.

Jane's actionable insights and event attendance are tracked and monitored against Quality of Life scores. Then the instant invention AI makes recommendations on how to improve Jane's experience in the community while also providing a tangible improvement to her quality of life scores.

Jane's data along with all of the other residents' data is pulled together to provide communities a dashboard allowing them to make community wide decisions.

The instant invention can be modified to capture information with regards to any businesses looking to collect data on residents such as senior living communities, corporations, local housing communities, colleges, universities, etc.

As is apparent the instant invention is innovative in that it does more than identify that there is a problem but takes the guesswork out of how to solve the problem by providing actionable insights that fit seamlessly into the staff workflow.

An example of how the system provides information for the caregivers so they can provide enhanced service to a resident can be best understood by the following example.

Jane Smith scored low on the creativity area of her Quality of Life (QoL) score. The instant invention system is then flagged to make recommendations to improve the creativity area of QoL score. The instant invention system also identifies that Jane has an interest in painting and that there is a painting activity scheduled on the community calendar for 2 μm. later that week. The system creates an actionable insights message prompting a staff member at the community to remind Jane that this event is happening. A typical message could be, “Jane Smith is interested in painting. Remind her about the Paint by Numbers event today at 2 p.m. in the Activities Room and ask her if she would like to attend.”

The goal of the recommendation is to encourage Jane to attend the event thereby giving her the opportunity to re-explore some of her creative interests. The actionable insight message is then automatically scheduled to be delivered the morning of the day of the event which is also shown on the calendar and the delivery of the message is assigned to the staff member who is scheduled to visit her. Once the insight has been delivered by the staff member, the staff member is prompted to select from a Likert scale how well the actionable insight was received by the resident. A Likert scale is a five (or seven) point scale which is used to allow the individual to express how much they agree or disagree with a particular statement.

The goal is that repeated completion and success of the insights will lead to Jane feeling more connected to the community and satisfied with her creative interests which will improve her overall Quality of Life.

Another example, John Smith took a wellness assessment that identified a need to be more physically active. The system identifies this need and makes the following recommendation: “Mr. Smith may enjoy going for a walk today”. The instant invention system will coach the caregiver to tell Mr. Smith about the weather and recommend that he invite residents Dan Morgan and Peter Johnson to go with him. The instant invention system identified that Mr. Smith used to go walking every morning but has not recently and that Mr. Smith prefers small group activities over large group activities. The instant invention system then selects from the resident matching feature the top two residents that John has the most in common with. However, any reasonable number of candidates is possible and that number is selectable by the staff. The instant invention can also scan various sources for weather information such as inclement weather in the area to alert the staff so they can modify a recommendation that depends on weather conditions. With these data points, the system makes a tailored actionable insight with the goal of encouraging John to participate in physical activities. Once the insight has been delivered by the staff member, the staff member is prompted to select from a Likert scale how well the actionable insight was received by the resident. Repeated completion and success of the actionable insight could lead to Mr. Smith feeling healthier, being more physically active and possibly building a new routine and new friendships.

Another example could be that resident Margaret Parker indicated in a satisfaction survey that there was an issue/need such as temperature of her room. The instant invention will make a recommendation to the staff to help the staff be more attentive to this need by sending an actionable insight message such as: “Margaret Parker has indicated dissatisfaction with the temperature of her room. When visiting her, ask if adjusting the temperature setting would make her more comfortable and adjust the thermostat accordingly.” Once the insight has been delivered by the staff member, they are prompted to select from a Likert scale how well the actionable insight was received by the resident.

The instant invention is a system for supporting an intelligent data collection system which utilizes audio transcription of a media files, artificial intelligence with video and pictorial integration.

The instant invention comprises the following components:
a. Data Collection using a media file audio recordings;
b. Artificial Intelligence module for transcribing the audio information of a media file;
c. Speech-to-text Application Programming Interface;
d. Smart Forms.
e. Digital Resident Profile module;
f. Resident Matching module;
g. Recommendation Engine module;
h. Inclusion Score module;
i. Data and Feedback module;
j. Dashboards and analytics module;

A first embodiment of the instant invention can be described as a resident profile system for residents in a senior living facility for determining actionable insights and recommending personalized care. The instant invention incorporates the following:
a. a smart mobile device with an IP address having a smart mobile device application, a smart mobile device application recording module and a smart mobile device data display module;
b. a cloud application or local server with a local server software system;
c. the smart mobile device with an IP address being in communication with a cloud application or local server; and
d. the smart mobile device application recording at least one media file audio recording.

The cloud application or local server with a local server software system is in communication with the smart mobile device and the cloud application or local server software system further has the following subsystems:
a. a data collection module;
b. an artificial intelligence module;
c. a speech-to-text API;
d. a smart forms module;
e. a digital resident profile module enabled to transmit resident profile data to the smart mobile device and the smart mobile device is enabled to use the smart mobile device data display module to display the resident profile data;
f. a resident matching module enabled to transmit resident matching data to the smart mobile device and the smart mobile device is enabled to use the smart mobile device data display module to display the resident matching data;
g. a recommendation engine module enabled to transmit resident recommendation data to the smart mobile device and the smart mobile device is enabled to use the smart mobile device data display module to display the resident recommendation data;
h. an inclusion score module enabled to transmit resident inclusion data to the smart mobile device and the smart mobile device is enabled to use the smart mobile device data display module to display the resident inclusion data;
i. a data and feedback module enabled to transmit data to the smart mobile device and receive feedback from the smart mobile device; and
j. a dashboard and analytics module,

The method of using the first embodiment of the instant invention can be best described as follows. To ensure adequate security the instant invention can utilize a file security system that such as a virtual private network (VPN) that enables users to send and receive data across shared or public networks as if their computing devices were directly connected to the private network.

The user of the first embodiment uses a smart mobile device with an IP address having a smart mobile device application to record at least one media file containing an audio recording and the user enters a password into the smart mobile device application and the a smart mobile device application transmits the password to the cloud application or local server with a local server software system and the cloud application or local server with a local server software system confirms the password to the smart mobile device with an IP address the session is allowed and the smart mobile device with an IP address transmits the at least one media file audio recording to the cloud application or local server with a local server software system else if the cloud application or local server with a local server software system does not confirm the password to the smart mobile device with a unique IP address then the smart mobile device with a unique IP address cannot transmit the at least one media file audio recording to the cloud application or local server with a local server software system and cloud application or local server with a local server software system ends the smart mobile device with a unique IP address to the cloud application or local server communication.

If the session is allowed then the local server smart mobile device is enabled to transfer the at least one media file audio recording to the on the cloud application or local server with a local server software system data collection module.

The data collection module is enabled to transfer the at least one media file audio recording to the artificial intelligence module.

The artificial intelligence module is enabled to receive the least one media file audio recording and transfers the least one media file audio recording using the speech-to-text API to a remote computer module. The remote computer receives the least one media file audio recording from the speech-to-text API and the remote computer module transcribes information from the at least one media file audio recording to an audio recording text file. The remote computer module transfers the audio recording text file to the speech-to-text API and the speech-to-text API transfers the audio recording text file to the artificial intelligence module and the artificial intelligence module is in communication the smart forms module.

The artificial intelligence module edits the audio recording text file and creates a corrected text file.

The artificial intelligence module is enabled to transfer the corrected text file to the smart forms module and creates smart forms data.

The digital resident profile module uses the smart forms data to display resident profile data on the smart mobile device.

The resident matching module is enabled to transfer the smart forms data to display resident matching data on the smart mobile device.

The recommendation engine module is enabled to use the smart forms data to display the patient recommendations on the smart mobile device using the smart mobile device data display module.

The inclusion score module is enabled to use the smart forms data to display the resident inclusion data on the smart mobile device using the smart mobile device data display module.

A second embodiment of the instant invention can be described as a resident profile system for residents in a senior living facility for determining actionable insights and recommending personalized care comprising:
a. a smart mobile device with a unique IP address (or dedicated device) having a smart mobile device application using at least one media file audio recording and a smart mobile device data display module;
b. a cloud application or local server with local server software system with a smart mobile verification system;
c. and the smart mobile device with a unique IP address being in communication with a cloud application or local server.
d. the smart mobile device application recording at least one media file audio recording.

The cloud application or local server with a local server software system is in communication with the smart mobile device with a unique IP address (or dedicated device) and the cloud application or local server software system comprising:
a. a smart mobile verification system;
b. a data collection module;
c. an artificial intelligence module;
d. a speech-to-text API;
e. a smart forms module;
f. a digital resident profile module enabled to transmit resident profile data to the smart mobile device and the smart mobile device is enabled to use the smart mobile device data display module to display aid resident profile data;
g. a resident matching module enabled to transmit resident matching data to the smart mobile device and the smart mobile device is enabled to use the smart mobile device data display module to display the resident matching data;
h. a recommendation engine module enabled to transmit resident recommendation data to the smart mobile device and the smart mobile device is enabled to use the smart mobile device data display module to display the resident recommendation data;
i. an inclusion score module enabled to transmit resident inclusion data to the smart mobile device and the smart mobile device is enabled to use smart mobile device data display module to display the resident inclusion data;
j. a data and feedback module enabled to transmit data to the smart mobile device and receive feedback from the smart mobile device; and
k. a dashboards and analytics module,

The method of using second embodiment of the instant invention can be best described as follows. A user of the smart mobile device with a unique IP address having a smart mobile device application records the at least one media file audio recording and the user enters a password and the smart mobile device with a unique IP address having a smart mobile device application transmits the unique IP address and the password to the cloud application or local server with local server software and the cloud application or local server with local server software uses the smart mobile verification system and confirms the password to the smart mobile device with a unique IP address. The smart mobile device with a unique IP address transmits the at least one media file audio recording to the cloud application or local server with local server software system. If the smart mobile verification system does not confirm the password to the smart mobile device with a unique IP address and the smart mobile device with a unique IP address cannot transmits at least one media file audio recording to the cloud application or local server with local server software system and cloud application or local server with a local server software system ends the smart mobile device with a unique IP address to the cloud application or local server communication.

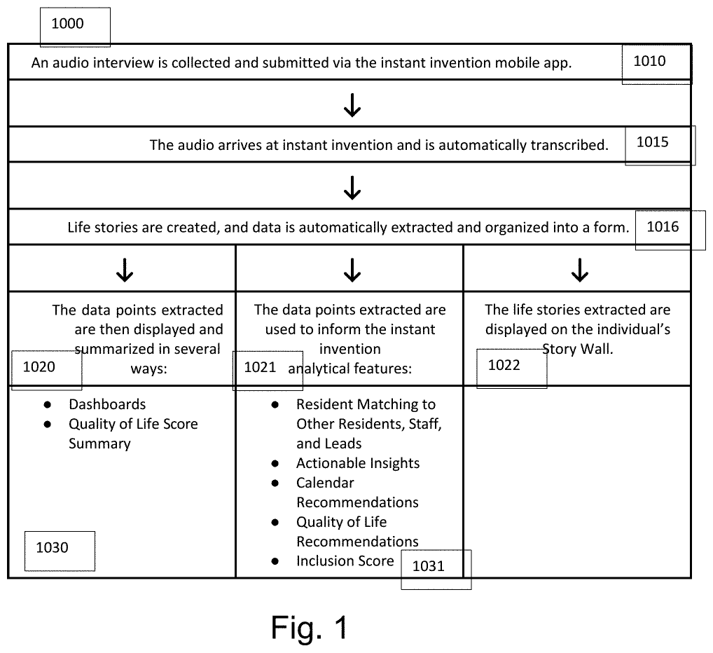

Referring now to the drawings shown in FIGS. 1-15, and more particularly to FIG. 1, there is shown a high level flow chart of the instant invention, method 1000. Step 1010 is the start of the method 1000 of the instant invention and an audio interview which is captured on the appropriate media file. The media file is collected and submitted via the instant invention mobile application. Next in Step 1015 the media file audio file arrives at instant invention and is automatically transcribed. Then at Step 1016 the instant invention creates Life stories, and data is automatically extracted and organized into a smart form. Once the data is organized into a smart form there are three possible routes for the data. Step 1020 the data points are extracted are then displayed and summarized in several ways which are shown in Step 1030 Dashboards and quality of life summary. An alternative is Step 1021 the data points extracted are used to inform the instant invention analytical features shown in Step 1031 and include Resident Matching to Other Residents, Staff, and Leads, Actionable Insights, Calendar, Recommendations, Quality of Life, Recommendations and Inclusion Score. The third option is shown in Step 1022 the life stories extracted are displayed on the individual's Story Wall.

The method of the instant invention and the resulting software can use a personal mobile device but a preferred embodiment is to load the instant invention on a dedicated mobile or smart device which can be connected to the cloud or a local server to protect the sensitive personal data of the residents.

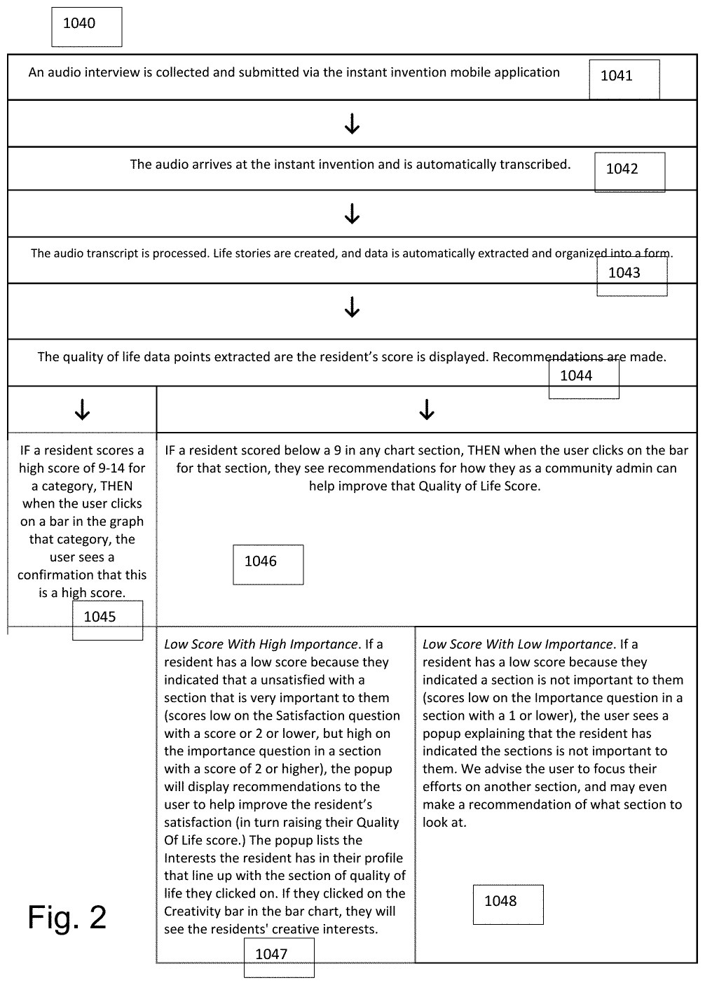

Referring to FIG. 2, there is shown method 1040 the instant invention Quality of Life Recommendations flow chart. Step 1041 an interview conducted and a media file captured including an audio file and submitted via the instant invention mobile application. In Step 1042 the audio portion of the media file is isolated and when it arrives at the instant invention it is automatically transcribed. Then the audio file is transferred to Step 1043 where the audio transcript is processed. Life stories are created, and data is automatically extracted and organized into a smart form. The instant invention in Step 1044 provides a method for the instant invention to extract the quality of life data points, the resident's score is displayed and recommendations are made. This presents two paths for the data. The first is Step 1045, IF a resident scores a high score of 9-14 for a category, THEN when the user clicks on a bar in the graph for that category, the user sees a confirmation that this is a high score. The second is Step 1046, IF a resident scored below a 9 in any chart section, then when the user clicks on the bar for that section, they see recommendations for how they, as a community administrator can help improve that Quality of Life Score. Step 1046 can result in two possible outcomes, Step 1047 Low Score with High Importance or Step 1048 Low Score with Low Importance. In Step 1047 Low Score with High Importance, If a resident has a low score and they indicated that they are unsatisfied with a section that is very important to them (for example, they produce a low score on the Satisfaction question with a score or 2 or lower, but high on the importance question in a section with a score of 2 or higher), the popup will display recommendations to the user to help improve the resident's satisfaction (in turn, raising their Quality of Life score.) The popup lists the Interests the resident has in their profile that line up with the section of quality of life they clicked on. If they clicked on the Creativity bar in the bar chart, they will see the residents' creative interests. The second alternative is Step 1048 Low Score with Low Importance. If a resident has a low score because they indicated a section is not important to them (scores low on the Importance question in a section with a 1 or lower), the user sees a popup explaining that the resident has indicated the section is not important to them, then we advise the user to focus their efforts on another section, and may even make a recommendation of what section to look at.

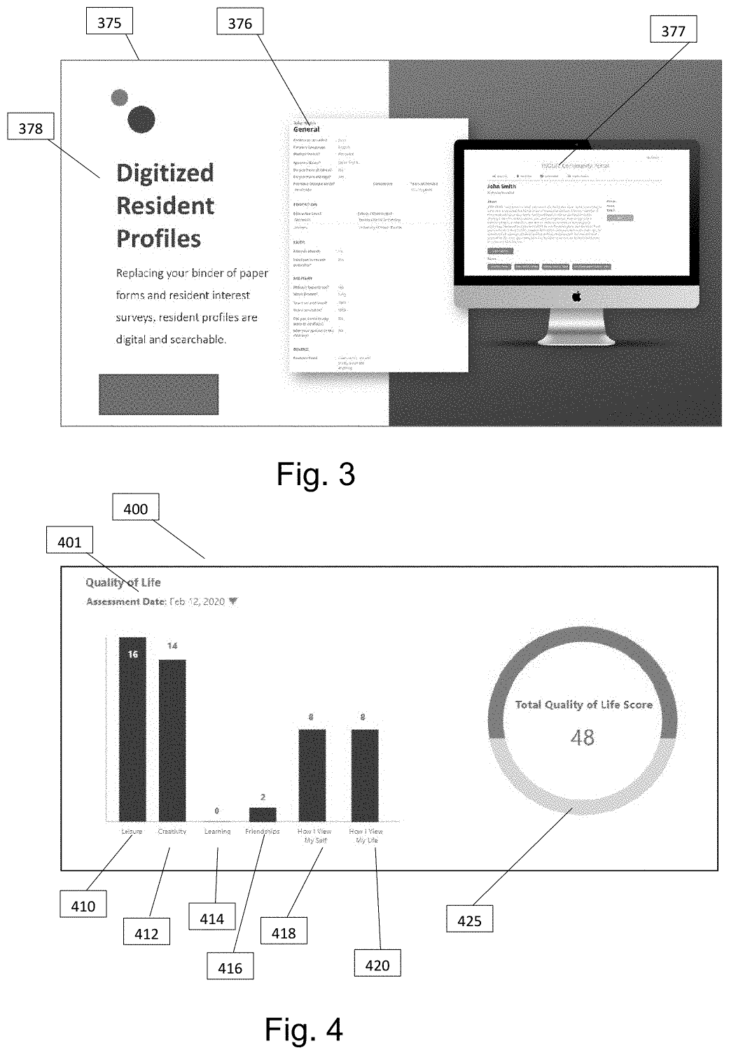

FIG. 3 shows a digitized resident profile screen 375 of the instant invention. The resident profile screen 375 displays the page identifier 378, general information about the resident and access to the community portal 377.

FIG. 4 shows a typical quality of life data screen 400. This screen provides the Quality of Life assessment date 401 and the scores for each component of the QoL score Leisure 410, Creativity 412, Learning 414, How the resident views themselves 418 and How the resident views their life 420. This components are summarized and displayed in a Total Quality of Life Score 425.

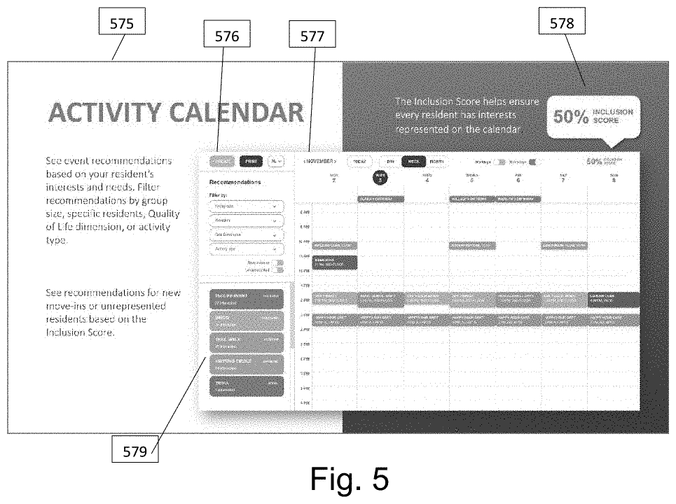

FIG. 5 shows a typical activity calendar data screen 575. This screen provides the Activity Calendar for the resident with recommendations based on the resident's interest and needs. The screen provides a method to create or print the calendar 576, the calendar 577 with activities noted by day and time 577 and the resident inclusion score 578. Section 579 of the activity calendar shows the color code and index of the activities such as bingo, trail walk or knitting circle. The activities are definable by the facility administrators so that each facility can customize events for their particular residents.

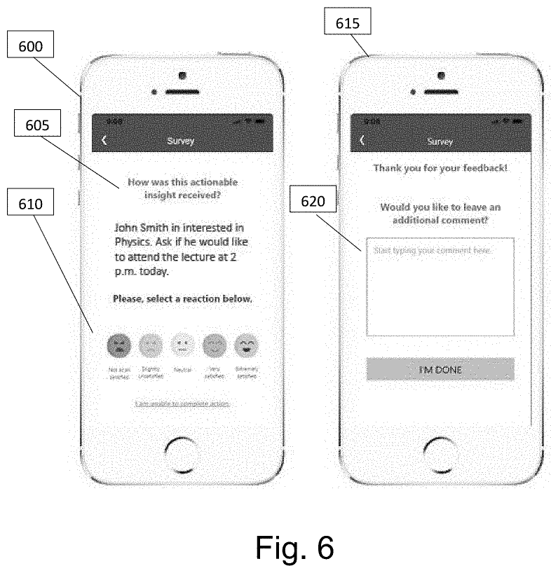

FIG. 6 shows a typical screens for feedback survey mechanism for actionable insights. The first screen 600 allow the caregiver to receive an actionable insight 605 and 610 allows the caregiver to provide feedback as to how the resident responded to the actionable insight. The second screen 615 provides a comment field 620 for the caregiver to provide additional information.

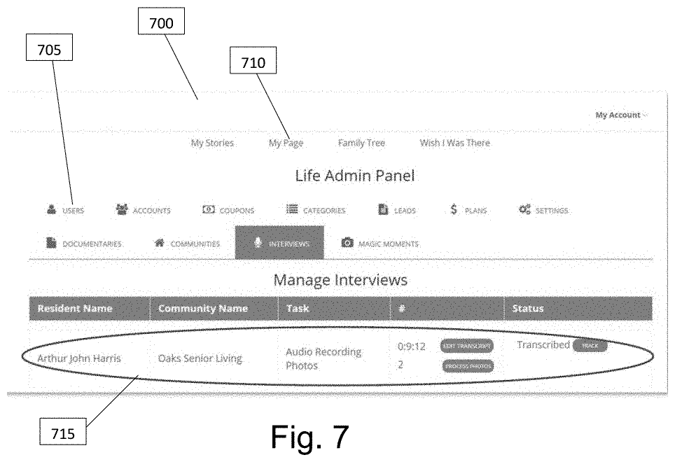

FIG. 7 shows a typical screen 700 for backend audio recordings which are capture as part of the media file and photos that are submitted in the mobile application and appear in the instant invention workflow. Menu 705 allows the user to navigate through the users, accounts, coupons , categories, leads, plans, settings, documentaries, communities, interviews and magic moments. The 710 menu allows the user to navigate through the Resident stories, Page, Family tree and wish I was there information page. The 715 field shows the interview and the details including audio and photos that are collected and allows the user to transcribe the audio files and load them to smart forms.

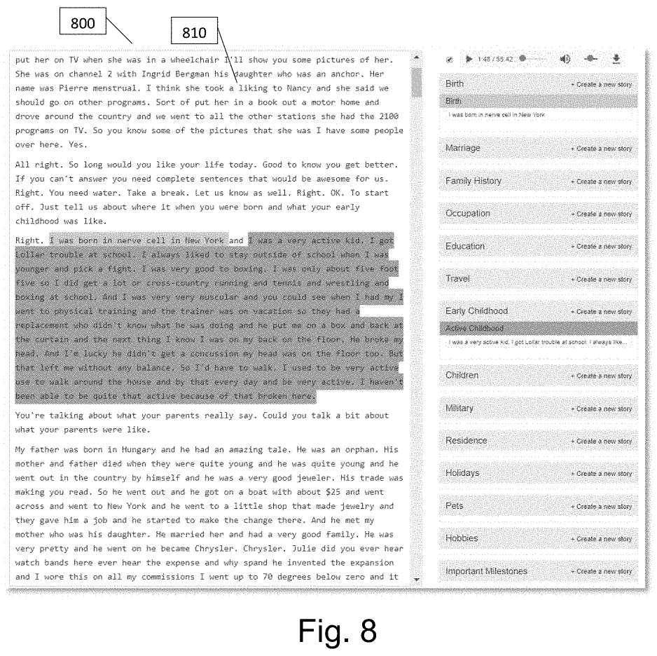

FIG. 8 shows the typical screen 800 for the backend of the instant invention showing the edit transcript 810 function and how the audio file can then be cut into individual stories.

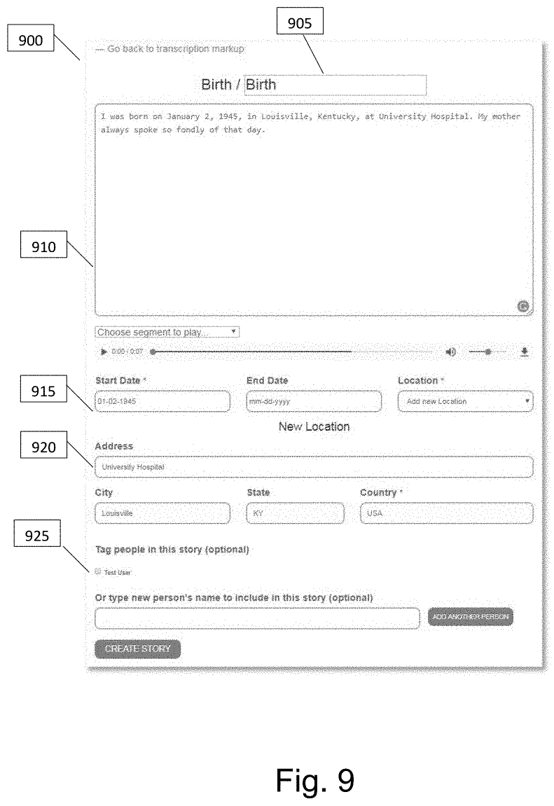

FIG. 9 shows the typical screen 900 for backend of the instant invention showing the stories are individually reviewed and edited to ensure quality. Item 905 is the input field for the event such as birth, marriage or high school graduation. Filed 910 is the input field for the data about the event. Field 915 is the start and end date as well as the location activation pull down menu. Field 920 is the address of the location and Field 925 allows the user to Tag other people in the story. This is a smart form and can be automated filled in from the audio file and edited by the user.

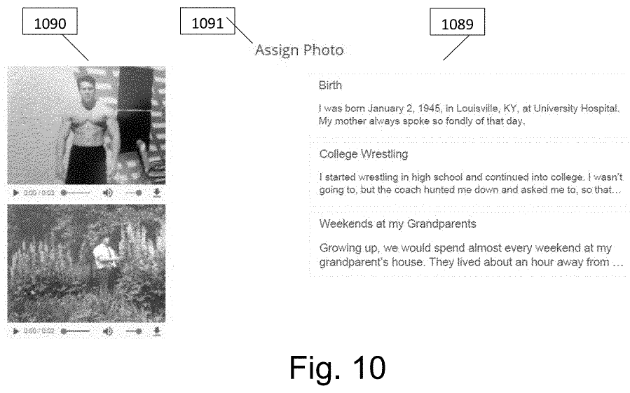

FIG. 10 shows the typical screen 1089 for backend of the instant invention showing that the stories 1091 are matched with photos 1090.

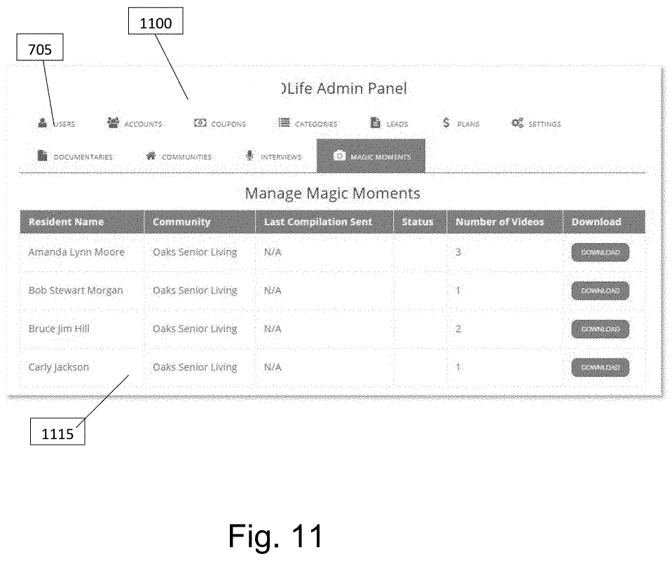

FIG. 11 shows the Life Administration screen 1100 where videos submitted through the mobile app appear in the instant invention workflow. Menu 705 allows the user to navigate through the users, accounts, coupons, categories, leads, plans, settings, documentaries, communities, interviews and magic moments. Field 1115 shows the Resident name and the magic moments videos associated with a Resident. This screen also allows the caregiver to download the information.

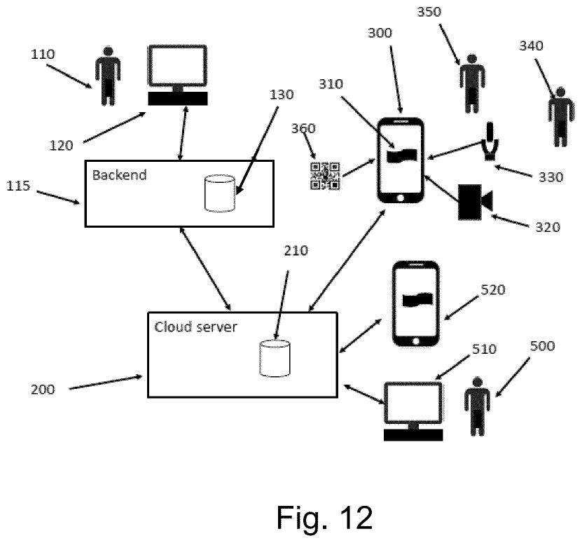

FIG. 12 shows schematic of the instant invention. The interviewer 350 reads the tag 360 prior to initiating the interview using the specialized mobile device 300 and the mobile application 310 and retrieves any information stored in the cloud or secure server 200 application 210 associated with tag 360. The interviewer 350 and interviewee 340 using the specialized mobile device 300 with mobile application 310 record media files comprising of audio and video information using microphone 330 and video camera 320. The video information can be in the form of video files or picture files. The mobile application then saves the interview data and transfers it either wirelessly or by wired connection to the cloud or secure server 200. The specialized mobile or smart device allows the user to access the information located on the cloud or on a secure server. By coupling the secure server to only specific mobile devoices the data is kept secure

The interview data transferred to the cloud or secure server 200 is stored in the instant invention application 210. The instant invention translates the audio information and fills in the forms and the interview data can then stores it in the instant invention application 210.

The back end system 115 and back end program 130 is connected to workstation 120 with operator 110. The operator 110 using the back end system 115, using back end program 130 and the workstation 120 edits the interview data.

The information can be viewed or edited by individual 500 using computer 510 or mobile device 520.

The cloud or secure server 200 limits the connectivity to the server by recognizing only specific IP addresses of the authorized specialized mobile device 300 and these require additional authorization such as password or security pin.

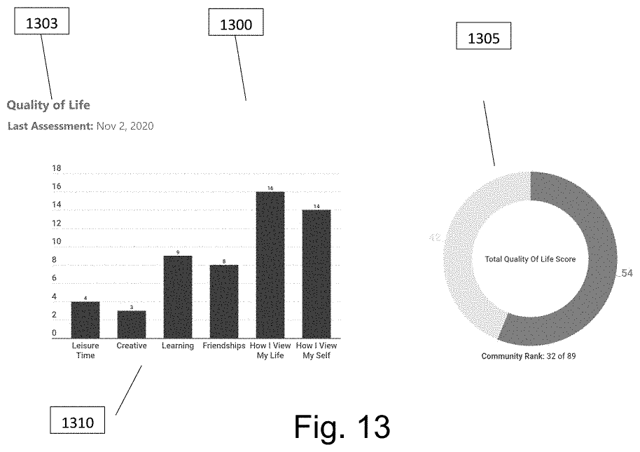

FIG. 13 shows a Quality of Life (QoL) summary screen 1300 for an individual resident. The instant invention technology is also used to collect assessments. In the instant invention intake interview, includes a validated Quality of Life scale, so residents are completing a baseline quality of life assessment as part of their first interview. These assessment responses are processed using our same smart form technology and stored as an assessment on the instant invention system. Assessment results can be viewed for each resident, and Quality of Life is displayed to the staff and at the communities visually for each resident and in aggregate across the community. Field 1303 is the QoL assessment date, field 1310 is the shows the specific QoL categories such as Leisure Time, Creativity, Learning, Friendships, How the resident views their life, how the resident views themselves. The graph 1305 provides the screen with an overall community Total QoL score.

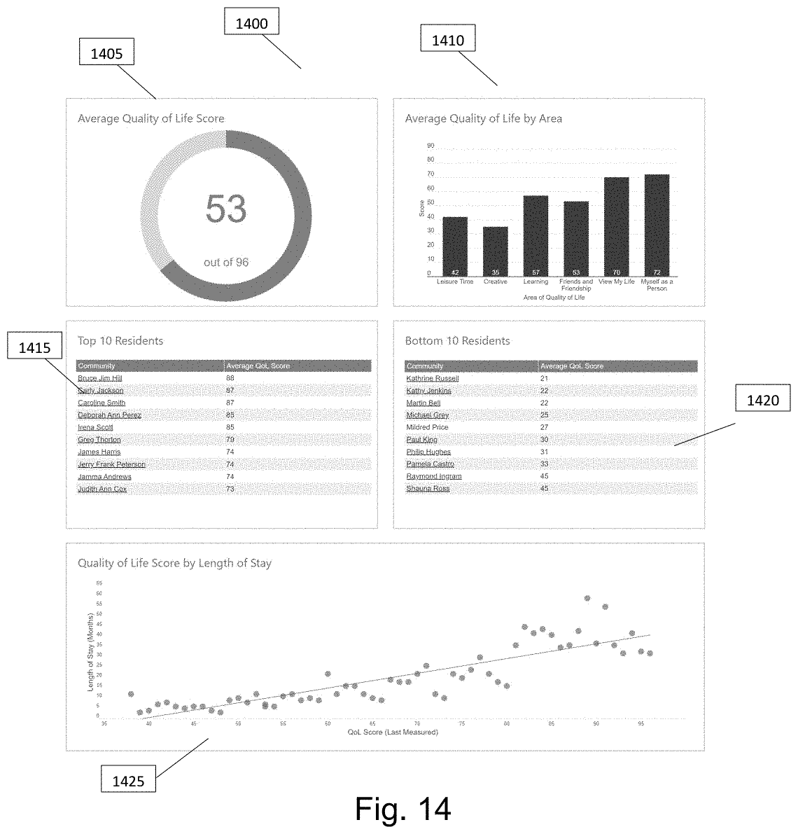

FIG. 14 shows a Community Quality of Life summary dashboard 1400. Field 1405 shows the average quality of life score for the facility in graphical form, field 1410 provides the average quality of life scores by category or area. Field 1415 provides the QoL scores of the top 10 Residents and field 1420 provides the QoL scores for the bottom 10 Residents. The spread between the top resident and bottom resident can be used as an indication of facility performance. Field 1425 is a graph the QoL score versus the length of stay of the Resident.

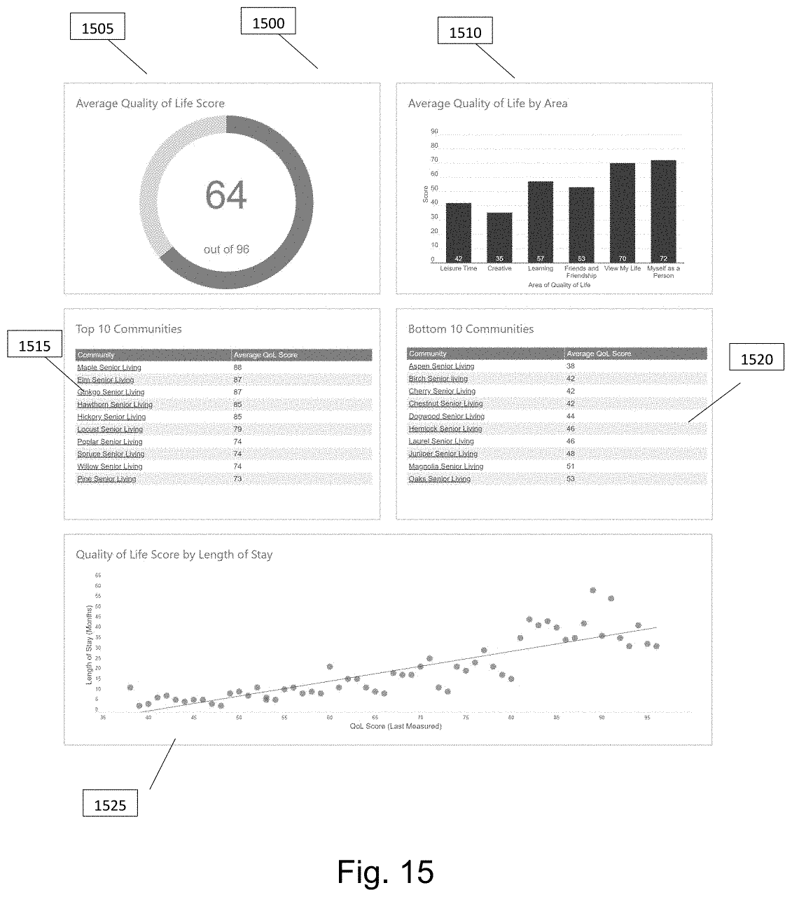

FIG. 15 shows a Corporate Quality of Life summary dashboard 1500. Field 1505 shows the average QoL score of the facility and field 1510 provides the average quality of life scores by category or area for the facility. Field 1515 provides the QoL scores of the top 10 facilities and field 1520 provides the QoL scores for the bottom 10 facilities. The spread between the top facility and bottom facility can be used as an indication of facilities overall performance with respect to their performance. Field 1525 is a graph the QoL score versus the length of stay of the Resident.

As noted, the cloud or secure server 200 limits the connectivity to the server by recognizing only specific IP addresses of the authorized specialized mobile device 300 and it also requires additional authorization such as password or security pin. My linking the cloud or secure server 200 applications and limiting the peer to peer transactions to the only transactions with specific dip address then the data has another level of security which is not afforded to an open cloud based system. The backend system or program 130 has specific IP addresses entered by the operator 110 to the back end system 115 and back end program 130. The system 115 and back end program 130 provides that information to the cloud or secure server 200 and the cloud or secure server 200 stores the specific IP addresses in the instant invention application 210. The cloud server will not communicate with a device which is not identified with a sanctioned IP address which thereby creates a more secure operating environment.

In some embodiments the method or methods described above may be executed or carried out by a computing system including a tangible computer-readable storage medium, also described herein as a storage machine, that holds machine-readable instructions executable by a logic machine (i.e. a processor or programmable control device) to provide, implement, perform, and/or enact the above described methods, processes and/or tasks. When such methods and processes are implemented, the state of the storage machine may be changed to hold different data. For example, the storage machine may include memory devices such as various hard disk drives, CD, or DVD devices. The logic machine may execute machine-readable instructions via one or more physical information and/or logic processing devices. For example, the logic machine may be configured to execute instructions to perform tasks for a computer program. The logic machine may include one or more processors to execute the machine-readable instructions. The computing system may include a display subsystem to display a graphical user interface (GUI) or any visual element of the methods or processes described above. For example, the display subsystem, storage machine, and logic machine may be integrated such that the above method may be executed while visual elements of the disclosed system and/or method are displayed on a display screen for user consumption. The computing system may include an input subsystem that receives user input. The input subsystem may be configured to connect to and receive input from devices such as a mouse, keyboard or gaming controller. For example, a user input may indicate a request that certain task is to be executed by the computing system, such as requesting the computing system to display any of the above described information, or requesting that the user input updates or modifies existing stored information for processing. A communication subsystem may allow the methods described above to be executed or provided over a computer network. For example, the communication subsystem may be configured to enable the computing system to communicate with a plurality of personal computing devices. The communication subsystem may include wired and/or wireless communication devices to facilitate networked communication. The described methods or processes may be executed, provided, or implemented for a user or one or more computing devices via a computer-program product such as via an application programming interface (API).

Since many modifications, variations, and changes in detail can be made to the described embodiments of the invention, it is intended that all matters in the foregoing description and shown in the accompanying drawings be interpreted as illustrative and not in a limiting sense. Furthermore, it is understood that any of the features presented in the embodiments may be integrated into any of the other embodiments unless explicitly stated otherwise. The scope of the invention should be determined by the appended claims and their legal equivalents.

In addition, the present invention has been described with reference to embodiments, it should be noted and understood that various modifications and variations can be crafted by those skilled in the art without departing from the scope and spirit of the invention. Accordingly, the foregoing disclosure should be interpreted as illustrative only and is not to be interpreted in a limiting sense. Further it is intended that any other embodiments of the present invention that result from any changes in application or method of use or operation, method of manufacture, shape, size, or materials which are not specified within the detailed written description or illustrations contained herein are considered within the scope of the present invention.

Insofar as the description above and the accompanying drawings disclose any additional subject matter that is not within the scope of the claims below, the inventions are not dedicated to the public and the right to file one or more applications to claim such additional inventions is reserved.

Although very narrow claims are presented herein, it should be recognized that the scope of this invention is much broader than presented by the claim. It is intended that broader claims will be submitted in an application that claims the benefit of priority from this application.

While this invention has been described with respect to at least one embodiment, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

What is claimed is:

1. A resident profile system for residents in a senior living facility for determining actionable insights and recommending personalized care comprising:
a. a smart mobile device with an IP address having a smart mobile device application, a smart mobile device application recording module and a smart mobile device data display module;
b. a cloud application or local server with local server software system;
c. and said smart mobile device with an IP address being in communication with said cloud application or local server with local server software system;
d. said smart mobile device application recording at least one media file audio recording; and
e. said smart mobile device application transmitting said at least one media file audio recording to said cloud application or local server with local server software system.

2. The resident profile system for residents in a senior living facility of claim 1 wherein said cloud application or local server with a local server software system is in communication with said smart mobile device and said cloud application or local server software system further comprising:
a. a data collection module;
b. an artificial intelligence module;
c. a speech-to-text API;
d. a smart forms module;
e. a digital resident profile module enabled to transmit resident profile data to said smart mobile device and said smart mobile device is enabled to use said smart mobile device data display module to display said resident profile data;
f. a resident matching module enabled to transmit resident matching data to said smart mobile device and said smart mobile device is enabled to use said a smart mobile device data display module to display said resident matching data;
g. a recommendation engine module enabled to transmit resident recommendation data to said smart mobile device and said smart mobile device is enabled to use said a smart mobile device data display module to display said resident recommendation data;
h. an inclusion score module enabled to transmit resident inclusion data to said smart mobile device and said smart mobile device is enabled to use said a smart mobile device data display module to display said resident inclusion data;
i. a data and feedback module enabled to transmit data to said smart mobile device and receive feedback from said smart mobile device; and
j. a dashboards and analytics module,

3. The method of using the resident profile system for residents in a senior living facility of claim 2 wherein a user of said smart mobile device with an IP address having a smart mobile device application records said at least one media file audio recording and said user enters a password into said smart mobile device application and said a smart mobile device application transmits said password to said cloud application or local server with a local server software system and said cloud application or local server with a local server software system confirms said password to said smart mobile device with an IP address and said smart mobile device with an IP address transmits said at least one media file audio recording to said cloud application or local server with a local server software system else if said cloud application or local server with a local server software system does not confirm said password to said smart mobile device with an IP address and said smart mobile device with a unique IP address cannot transmit said at least one media file audio recording to said cloud application or local server with a local server software system and end said smart mobile device with a unique IP address to said cloud application or local server communication.

4. The method of claim 3 wherein said cloud application or local server smart mobile device transfers said at least one media file audio recording to said data collection module.

5. The method of claim 3 wherein said data collection module transfers said at least one media file audio recording to said artificial intelligence module.

6. The method of claim 3 wherein said artificial intelligence module receives said least one media file audio recording and transfers said least one media file audio recording using said speech-to-text API to a remote computer module that receives said least one media file audio recording from said speech-to-text API and said remote computer module transcribes information from said at least one media file audio recording to an audio recording text file and said remote computer module transfers said audio recording text file to said speech-to-text API and said speech-to-text API transfers said audio recording text file to said artificial intelligence module and said artificial intelligence module is in communication said smart forms module.

7. The method of claim 3 wherein said artificial intelligence module edits said audio recording text file and creates a corrected text file.

9. The method of claim 3 wherein said artificial intelligence module transfers said corrected text file to said smart forms module and creates smart forms data.

10. The method of claim 3 wherein said digital resident profile module uses said smart forms data to display resident profile data on said smart mobile device.

11. The method of claim 3 wherein said resident matching module uses said smart forms data to display resident matching data on said smart mobile device.

12. The method of claim 3 wherein said recommendation engine module uses said smart forms data to display said patient recommendations on said smart mobile device using said smart mobile device data display module.

13. The method of claim 3 wherein said inclusion score module uses said smart forms data to display said resident inclusion data on said smart mobile device using said smart mobile device data display module.

14. A resident profile system for residents in a senior living facility for determining actionable insights and recommending personalized care comprising:
a. a smart mobile device with a unique IP address having a smart mobile device application using at least one media file audio recording and a smart mobile device data display module;
b. a cloud application or local server with a cloud application or local server software system with a smart mobile verification system;
c. and said smart mobile device with a unique IP address being in communication with a cloud application or local server.
d. said smart mobile device application recording at least one media file audio recording; and
e. said smart mobile device application transmitting said at least one media file audio recording to said cloud application or local server with local server software system.

15. The resident profile system for residents in a senior living facility of claim 14 wherein said cloud application or local server with a cloud application or local server software system is in communication with said smart mobile device and said cloud application or local server software system comprising:
a. a smart mobile verification system
b. a data collection module;
c. an artificial intelligence module;
d. a speech-to-text API;
e. a smart forms module;
f. a digital resident profile module enabled to transmit resident profile data to said smart mobile device and said smart mobile device is enabled to use said a smart mobile device data display module to display aid resident profile data;
g. a resident matching module enabled to transmit resident matching data to said smart mobile device and said smart mobile device is enabled to use said a smart mobile device data display module to display said resident matching data;
h. a recommendation engine module enabled to transmit resident recommendation data to said smart mobile device and said smart mobile device is enabled to use said a smart mobile device data display module to display said resident recommendation data;
i. an inclusion score module enabled to transmit resident inclusion data to said smart mobile device and said smart mobile device is enabled to use said a smart mobile device data display module to display said resident inclusion data;
j. a data and feedback module enabled to transmit data to said smart mobile device and receive feedback from said smart mobile device; and
k. a dashboard and analytics module,

16. The method of using the resident profile system for residents in a senior living facility of claim 15 wherein a user of said smart mobile device with a unique IP address having a smart mobile device application records said at least one media file audio recording and said user enters a password and said smart mobile device with a unique IP address having a smart mobile device application transmits said unique IP address and said password to said cloud application or local server with a cloud application or local server software system and said cloud application or local server with a cloud application or local server software uses said smart mobile verification system and confirms said password to said smart mobile device with a unique IP address and said smart mobile device with a unique IP address transmits said at least one media file audio recording to said cloud application or local server with a cloud application or local server software system or if said smart mobile verification system does not confirm said password to said smart mobile device with a unique IP address and said smart mobile device with a unique IP address cannot transmits at least one media file audio recording to said cloud application or local server with a cloud application or local server software system and end said smart mobile device with a unique IP address to said cloud application or local server communication.
